(19) United States
(12) Patent Application Publication
(10) Pub. No.: US 2009/0318856 A1
(43) Pub. Date: Dec. 24, 2009

(54) Sampling Ocular Fluid

(76) Inventor: Bert M. Glaser, Baltimore, MD (US)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 12/337,602

(22) Filed: Dec. 17, 2008

Related U.S. Application Data
(63) Continuation-in-part of application No. PCT/US2008/007527, filed on Jun. 18, 2008.

(51) Int. Cl. A61M 1/00

(52) U.S. Cl. 604/35

(57) ABSTRACT

The present invention includes an ophthalmic aspirating device that allows the relatively non-invasive removal of small volumes of ocular fluid for diagnostic and other purposes. The invention also includes an ophthalmic aspirating device that allows the for the injection of materials into the eye at the time of aspiration. The invention further includes methods of aspirating ocular fluid from an eye using an ophthalmic aspirating device.

This application is a continuation-in-part of PCT/US2008/007527, filed on Jun. 18, 2008, the entirety of which is herein incorporated by reference.

### BACKGROUND OF THE INVENTION

Ocular diseases include diabetic retinopathy, cystoid macular edema, idiopathic choroidal neovascularization, age-related macular degeneration, epiretinal membrane, macular hole, retinal detachment, vitreous hemorrhage, epiretinal membrane, retinal angiomatous proliferation, proliferative diabetic retinopathy, choroidal neovascularization, glaucoma, cataract, and uveitis.

Diabetic retinopathy (DR) is the most prevalent cause of vision loss in working adults. Most patients with type 1 diabetes mellitus and over 60% of those with type 2 diabetes eventually develop retinal vascular abnormalities. 20% to 30% of these patients advance to active proliferative diabetic retinopathy (PDR) and/or diabetic macular edema. Increased retinal vascular permeability (RVP) is a primary cause of diabetic macular edema and a characteristic finding in PDR. Cystoid Macular Edema (CME) is a type of edema of the macula that causes retinal damage and occurs in a wide variety of ocular disorders. Age-related macular degeneration (AMD) is the leading cause of vision loss and blindness in individuals over age 60 in the developed world. The disorder is characterized by the loss of central vision caused by pathologic aging of the macula. The manifestations of the disease are classified into two forms: non-exudative (dry) and exudative (wet or neovascular). Early diagnosis and preventative treatments for these and other ocular disorders remain a major unmet clinical need.

Efforts have been made to identify biomarkers from ocular vitreous fluid in an attempt to diagnosis ocular diseases. However, diagnostic sampling of vitreous fluid has invariably involved invasive procedures such as pars plana vitrectomy that pose risks such as damage to the crystalline lens, accelerated cataract formation, retinal detachment, and vitreous hemorrhage. Vitrectomies have also traditionally involved the removal of a volume of fluid that can cause at least partial collapse of the eye and can necessitate introduction of air or fluid to compensate for the volume removed. These limitations of surgical vitrectomy point to the need for materials and methods for obtaining vitreous fluid in a less invasive, more convenient, and relatively pain free manner.

Aspects of the present invention solve these problems, as well as others, by providing the ability to quickly and easily remove small quantities of fluid from the eye of a patient without the need for irrigating the eye and replenishing the eye with an equivalent amount of fluid. Thus, among other things, the patient does not experience the discomfort and pain of having large syringes inserted into the eye, with the inherent danger of infection during the aspiration and irrigation cycle, and the described procedure can be performed as a routine outpatient surgical technique.

### SUMMARY OF THE INVENTION

The present invention includes methods and materials for a non-surgical method of predicting or monitoring the physiological state of the eye. The method comprises aspirating a sample of vitreous fluid from the eye of a living subject, where the aspirating is not concurrent to eye surgery.

One aspect of the invention comprises an ophthalmic aspirating device for aspirating an ocular fluid, comprising a conduit having an outside diameter between 23 and 27 gauge, an inside diameter between 0.0120 inch and 0.0140 inch, and a wall thickness less than or equal to 0.004 inches, where the conduit has a distal conduit end for insertion into an eye with a first aperture and with a beveled edge of between 25 degrees and 40 degrees, and the distal conduit end being in fluid flow communication with a proximal conduit end with a second aperture, and further comprising a negative pressure module with a stopper operably linked to the proximal conduit end, where upon application of pressure to the negative pressure module, the stopper is pressed against the proximal conduit end causing the proximal conduit end to puncture the stopper resulting in a vacuum of between 500 mmHg and 1500 mmHg in the negative pressure module thereby aspirating between 10-200 microliters of ocular fluid into the negative pressure module.

In another aspect, the invention comprises an ophthalmic aspirating device for aspirating an ocular fluid, comprising a conduit having an outside diameter between 23 and 27 gauge, an inside diameter between 0.0120 inch and 0.0140 inch, and a wall thickness less than or equal to 0.004 inches, wherein the conduit has a distal conduit end for insertion into an eye with a first aperture and with a beveled edge of between 25 degrees and 40 degrees, and the distal conduit end being in fluid flow communication with a proximal conduit end with a second aperture and a negative pressure module operably linked to the proximal conduit end capable of creating a vacuum of between 500 mmHg and 1500 mmHg for aspiration of between 10-200 microliters of ocular fluid.

In another aspect, the invention comprises an ophthalmic aspirating device for aspirating an ocular fluid, comprising a tube having a first end and a second end and a first conduit disposed on the first end of the tube and having an outside diameter between 23 and 27 gauge, an inside diameter between 0.0120 inch and 0.0140 inch, a wall thickness less than or equal to 0.004 inches, a proximal end, and a distal end, where the proximal end is operably connected to the first end of the tube and the distal end is positioned to enable insertion inside an eye and has a first aperture with a beveled edge of between 25 degrees and 40 degrees and a second conduit disposed on the second end of the tube and having a near end and a far end, where the near end is operably connected to the second end of said tube, and a negative pressure module is disposed on the far end of the second conduit, where the negative pressure module is set for 1000 mmHg and where upon the insertion inside the eye of the first conduit, the negative pressure module has reduced pressure relative to pressure in the eye thereby creating a vacuum of between 500 mmHg and 1500 mmHg, and the vacuum generates an aspirating action that is transmitted through the tube and via the first conduit into the eye resulting in aspiration of between 10 - 200 microliters of vitreous fluid from the eye when the distal end of the conduit for 30 second, through the first conduit, into the tube.

In yet another aspect, the invention comprises an ophthalmic aspirating device for aspirating an ocular fluid, comprising a tube having a first end and a second end, a conduit disposed on the first end of the tube, where the conduit has an outside diameter between 23 and 27 gauge, an inside diameter between 0.0120 inch and 0.0140 inch, and a wall thickness less than or equal to 0.003 inches, the conduit further has a proximal end and a distal end, where the proximal end is operably connected to the first end of the tube and the distal end is positioned to enable insertion inside an eye and has an aperture and a beveled edge of between 25 degrees and 40 degrees, and a plunger disposed on the second end of the tube, a biasing mechanism operably connected to the plunger and having an actuator for controlling the biasing mechanism, where the biasing mechanism, when activated by the actuator, pulls the plunger away from the conduit and creates a vacuum of between 500 mmHg and 1500 mmHg, resulting in aspiration of between 10-200 microliters of vitreous fluid from an eye, through the conduit, into the tube.

In another aspect, the invention comprises a dual bore needle for aspirating ocular fluid and for injecting materials into an eye, comprising an outer conduit for insertion into an eye, the outer conduit having an outside diameter between 23 and 27 gauge, and a wall thickness less than or equal to 0.004 inches, an aperture, and a beveled edge of between 25 degrees and 40 degrees, and an inner tube for injecting materials into the eye, the inner tube having an outside diameter between 30 gauge and 40 gauge, and a wall thickness less than or equal to 0.004 inches, the inner tube disposed in the center of the outer conduit beginning 1 mm or more behind the aperture of the outer conduit. In another aspect the inner tube disposed in the center of the outer conduit beginning 0.5 mm or more behind the aperture of the outer conduit.

In yet another aspect, the invention comprises a conduit for aspirating ocular fluid, comprising a needle having an edge beveled between 25 and 40 degrees, the edge comprising an outer edge and an inner edge, the outer edge located at a distal most end of the needle, the outer edge further sharpened, and the inner edge further sharpened.

In another aspect, the invention comprises a method for aspirating vitreous fluid from an eye comprising inserting a distal end of a conduit into the eye, where the distal conduit end comprises a first aperture with a beveled edge of between 25 and 40 degrees, and the conduit has an outside diameter between 23 and 27 gauge, an inside diameter between 0.0120 inch and 0.0140 inch, and a wall thickness less than or equal to 0.002 inches, applying pressure to a negative pressure module containing a stopper, where the negative pressure module is operably associated with a proximal end of the conduit containing an aperture, causing the stopper to press against the proximal end of the conduit, thereby puncturing the stopper, creating a vacuum in the negative pressure module between 500 mmHg and 1500 mmHg, and withdrawing between 10-200 microliters of vitreous fluid from the eye into the negative pressure module.

In yet another aspect, the invention comprises a method for aspirating liquid from an eye comprising inserting a first conduit into an eye, where the first conduit has an outside diameter between 23 and 27 gauge, an inside diameter between 0.0120 inch and 0.0140 inch, a wall thickness less than or equal to 0.004 inches, an aperture, a beveled edge of between 25 degrees and 40 degrees, and where the first conduit is connected to a first end of a tube, opening a valve, where the valve is located on a second end of the tube, which is connected to a negative pressure module via a second conduit, and where the opening allows the tube to communicate, via the second conduit, with the negative pressure module, resulting in a vacuum of between 500 mmHg and 1500 mmHg inside the negative pressure module, creating an aspiration force inside the tube, and aspirating between 10-200 microliters of vitreous fluid from the eye into the tube.

In another aspect, the invention comprises a method for aspirating ocular fluids and for injecting materials into an eye comprising inserting an outer conduit into the eye, where the outer conduit comprises an aperture, a beveled edge of between 25 and 40 degrees, the outer conduit having an outside diameter between 23 and 27 gauge, and a wall thickness less than or equal to 0.004 inches, applying a vacuum of between 500 mmHg and 1500 mmHg, aspirating between 10-200 microliters of vitreous fluid from the eye, injecting materials into the eye by releasing the materials through an inner tube having an outside diameter between 30-40 gauge, and a wall thickness less than or equal to 0.004 inches, the inner tube disposed in the center of the outer conduit beginning 1 mm or more behind the aperture of the outer conduit.

### BRIEF DESCRIPTION OF THE DRAWINGS

FIG. 1 illustrates an ophthalmic aspiration device.

FIG. 2 illustrates an ophthalmic aspiration device.

FIG. 3 illustrates an ophthalmic aspiration device.

FIG. 4 illustrates a dual bore needle.

FIG. 5 illustrates a conduit with a beveled edge.

### DETAILED DESCRIPTION

The present invention provides and includes methods and materials for a non-surgical method of predicting or monitoring the physiological state of the eye. A method that includes aspirating a sample of vitreous fluid from the eye of a living subject, where the aspirating is not concurrent to eye surgery is also provided.

In an aspect, ocular fluids can be removed from a subject in need thereof using less invasive methods and instrumentation than a whole-bore vitrectomy cannula or cutter. In accordance with an aspect of this invention, an ophthalmic aspirating device is included in the present invention. In one aspect, an ophthalmic aspirating device for aspirating an ocular fluid comprises a conduit having an outside diameter between 23 and 27 gauge, an inside diameter between 0.0120 inch and 0.0140 inch, and a wall thickness less than or equal to 0.004 inches, where the conduit has a distal conduit end for insertion into an eye with a first aperture and with a beveled edge of between 25 degrees and 40 degrees, and the distal conduit end being in fluid flow communication with a proximal conduit end with a second aperture; and a negative pressure module with a stopper operably linked to the proximal conduit end, where upon application of pressure to the negative pressure module, the stopper is pressed against the proximal conduit end causing the proximal conduit end to puncture the stopper resulting in a vacuum of between 500 mmHg and 1500 mmHg in the negative pressure module aspirating between 10-200 microliters of ocular fluid into the negative pressure module. In a preferred aspect, an ocular fluid is vitreous fluid.

Any ocular or eye-related fluid can be analyzed in accordance with the present invention, including, e.g., vitreous fluids; anterior fluids, aqueous fluids; retinal blood, such as blood present in the choroid; and tears, including tears extracted from the lacrimal sac. In some cases the state of specific diseases as reflected in ocular fluids can be measured by fluorescent, magnetic, or radio nucleotide imaging. In a preferred aspect, vitreous fluid can be or can not be contaminated with vitreous gel.

In an aspect of the present invention, the amount of a fluid sample collected from a human eye has no cells. In another aspect, the volume of cells is 1%, 2%, 3%, 5%, or less than 10% of the total volume. In another aspect of the present invention, there are fewer than 10, 20, 50, 75, 100, 1000, or 5000 cells in the fluid sample.

In one aspect, a conduit is a needle or a cannula. Needles and other conduits can have apertures at their ends. One or more of these ends can be sharp to allow puncture of the eye, puncture of a container such as a negative pressure module, or puncture of both. In one aspect, a conduit has an outside diameter to allow self sealing scleral incision either through the pars plana or elsewhere on the eye. In another aspect, a conduit has an outside diameter of between 23 to 26, 24-25 gauge, or 25-26 gauge more preferably, the gauge is 23, 24, 25, or 26, most preferably the gauge is 25. In another aspect, the outside diameter of a conduit is less than 0.300 inches, between 0.0100-0.0300 inches, more preferably between 0.0150-0.0250 inches, most preferably 0.0.0200 inches. A conduit can be any length. A preferred length is less than one inch, more preferably ⅝ inches.

In a preferred aspect, a conduit has fluid flow communication throughout. In another aspect, a conduit has fluid flow communication throughout the conduit and is operably linked to a tube. In one aspect, an inside diameter of a conduit is between 0.0050-0.0250 inches, more preferably between 0.0100-0.0200, 0.0120-0.0140, most preferably 0.0130 inches. In one aspect, the conduit provides the thinnest wall and largest bore possible to increase sampling success and to reduce the outside diameter to maximize the self sealing of the wound. In another aspect, the conduit has a minimized wall thickness to allow reduction of outside diameter to maximize self sealing of scleral wound. In a preferred aspect, the wall thickness is less than or equal to 0.0040, about 0.0040, 0.0040-0.001, 0.0040-0.002, 0.0040-0.003, 0.0030-0.002, or preferably 0.0040-0.002 inches.

A sample is positive if greater than 50 microliters of ocular fluid is collected. A sample is labeled as “dry” if less than 50 microliters of ocular fluid is obtained. In a preferred aspect, the ocular fluid is vitreous fluid.

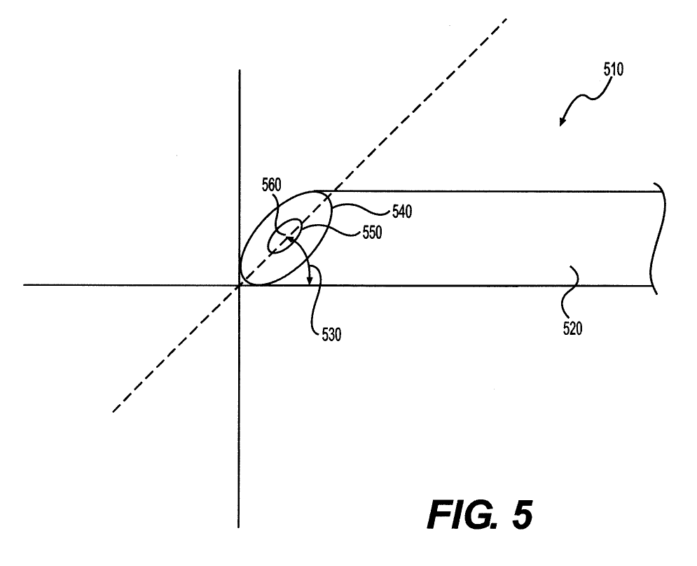

Further to the present invention, a conduit can have a distal end and a proximal end. The present invention also provides that distal and proximal conduit ends are in fluid flow communication. The present invention also includes a distal conduit end that can be inserted into an eye where there is a first aperture. A first aperture on a conduit end can have a beveled angle. In an aspect a first aperture on a conduit distal end can have a beveled angle. A beveled angle can be maximized to allow smooth entry of the distal conduit end into an eye thereby reducing patient discomfort and minimizing scleral wound size and allowing maximal self sealing of scleral wound. A bevel angle can be 20-45 degrees, preferably 25-40 degrees, about 30 degrees, or most preferably 30 degrees. The angle is measured from the center of the first aperture at the distal conduit end to the outside base of the conduit. See FIG. 5. The outer edge of the bevel can be sharpened to minimize patient discomfort and maximize ease of passage through the sclera. The inner edge of the bevel can be sharpened to cut vitreous when aspiration is performed under a vacuum thereby facilitating removal of ocular fluid. In a preferred aspect a conduit is 25 G×⅝″ (0.50×16 mm), supplied by Terumo.

A proximal conduit end can have a second aperture. In a preferred aspect, the proximal conduit end is operably linked to a negative pressure module with a stopper. Also optionally included in the present invention, a stopper is pressed against a proximal conduit end, it causes the proximal conduit end to puncture the stopper resulting in a vacuum.

In an aspect of the present invention, a distal conduit end is inserted inside of an eye and is in fluid flow communication with a negative pressure module. The present invention includes a negative pressure module that is capable of negative pressure relative to pressure in the eye thereby creating a vacuum of between 500-1500 mmHg, 750 mmHg-1200 mmHg and most preferably 1000 mmHg. A vacuum generates an aspirating action that is transmitted via a conduit or through a tube and via a first conduit into an eye resulting in aspiration of vitreous fluid from the eye. In one aspect, the vacuum applied is between 500 mmHg and 1500 mmHg. In another aspect, the vacuum applied is between 600 mmHg and 1400 mmHg. In another aspect, the vacuum applied is between 700 mmHg and 1300 mmHg. In yet another aspect, the vacuum applied is between 800 mmHg and 1200 mmHg. In still another aspect, the vacuum applied is between 900 mmHg and 1100 mmHg. In a preferred aspect, the vacuum applied is about 1000 mmHg.

In an aspect, the amount of sample size is determined by how long the vacuum is applied and how many mmHg are applied over that timeframe. In one aspect, the overall time course for taking a sample is less than 3 minutes, more preferably less than 2 minutes, such as between 30 seconds-2 minutes, preferably 30 seconds-1 minute, most preferably about 30 seconds. In an aspect, the application of a vacuum is controlled so that it can be increased according to a predetermined time course. In one aspect, the vacuum is increased linearly along a predetermined time course. In another aspect, the vacuum is increased non-linerally along a predetermined vacuum/time course. In another aspect, the vacuum is increased along a predetermined stepped vacuum/time course. For example, without limitation, the vacuum could be set at 1000 mmHg before the conduit is in the eye. Alternatively, the vacuum could be set to quickly be raised to 1000 mmHg after the conduit is in the eye. In another aspect, the vacuum could be brought up linearly to 1000 mmHg. Alternatively, the vacuum could be raised in steps, such as 100 mmHg each step with a step being determined by a time course, such as every second, or each 2 seconds, or 1.5 seconds. Any of the above methods of increasing the application of the vacuum can be accomplished with a user programmable vacuum/time course. In a preferred aspect, the vacuum/time course can be modified by a user at any time during its operation.

In another aspect, the application of a vacuum is controlled so that it can be decreased according to a predetermined time course. In one aspect, the vacuum is decreased linearly along a predetermined time course. In another aspect, the vacuum is decreased non-linearly along a predetermined vacuum/time course. In another aspect, the vacuum is decreased along a predetermined stepped vacuum/time course. For example, without limitation, the vacuum could be reduced quickly after the desired volume of ocular fluid is obtained. Alternatively, the vacuum could be set to slowly be reduced after reaching 1000 mmHg by a chosen time course to step down to 100-200 mmHg over a time course, such as every second, or each 2 seconds, or 1.5 seconds. In another aspect, the vacuum could be reduced linearly to 0 or linearly to a step such as 700 mmHg and then hold for 3 seconds. Any of the above methods of decreasing the application of the vacuum can be accomplished with a user programmable vacuum/time course. In a preferred aspect, the vacuum/time course can be modified by a user at any time during its operation.

In an aspect of the present invention, the amount of a fluid sample collected can be between 5 to 200 microliters, 10-200 microliters, 20-200 microliters and preferably 20-100 microliters. In a preferred aspect about 50 microliters of sample is obtained. In another aspect, less than or equal to 50 microliters, less than or equal to 100 microliters, less than or equal to 30 microliters is collected. In a further aspect, the amount of a fluid sample collected can be between 50-200, 50-100, 20-60, between 40-60, between 10-30, or between 10-40 microliters. In a preferred aspect, sampling can be repeated.

The aspiration rate of the ocular fluid is commensurate with the pressure of the vacuum inside the negative pressure module. The size, pressure, or size and pressure of the negative pressure module can be adjusted so as to aspirate a precise and desired amount of fluid. In a preferred aspect, an ocular fluid is vitreous fluid.

A vitreous sample to be analyzed in accordance with the present invention can be stored in any suitable way. For example, the sample can be extracted into a reservoir comprising at least one chemical to protect the polypeptide integrity. In some aspects, the chemical is a protease inhibitor or a phosphatase inhibitor.

Chemicals that can be included in a reservoir include, e.g., nucleic acids, protease inhibitors; phosphatase inhibitors; various preservatives, etc. Specific examples include, serine protease inhibitors, cysteine protease inhibitors, aspartic protease inhibitors, and metalloprotease inhibitors. Examples of these include, AEBSF, aprotinin, E-64, EDTA, leupeptin, bestatin, 0-phenanthroline, cathepsin, etc.

Also provided in the present invention is an ophthalmic aspirating device for aspirating an ocular fluid comprising a tube having a first end and a second end; a first conduit disposed on the first end of the tube and having an outside diameter between 23 and 27 gauge, an inside diameter between 0.0120 inch and 0.0140 inch, a wall thickness less than or equal to 0.004 inches, a proximal end, and a distal end, wherein the proximal end is operably connected to the first end of the tube and the distal end is positioned to enable insertion inside an eye and has a first aperture with a beveled edge of between 25 degrees and 40 degrees; a second conduit disposed on the second end of the tube and has a near end and a far end, where the near end is operably connected to the second end of the tube; and a negative pressure module is disposed on the far end of the second conduit, where the insertion inside the eye of the first conduit, the negative pressure module has reduced pressure relative to pressure in the eye thereby creating a vacuum of between 500 mmHg and 1500 mmHg, and the vacuum generates an aspirating action that is transmitted through the tube and via the first conduit into the eye resulting in aspiration of between 10-200 microliters of ocular fluid from the eye, through the first conduit, into the tube. In a preferred aspect, an ocular fluid is vitreous fluid.

In an aspect, a tube has a first and second end with fluid flow communication between the ends. A tube can be made out of any material. In a preferred aspect, a tube is made of plastic. A tube can be any length. In a preferred aspect, a tube can be a syringe. A preferred tube is a Beckton Dickinson 1 ml syringe (#309626). In another aspect, a tube can have markings or indentations on it to indicate, for example without limitation, volume. A tube can have any inner or outer diameter provided that a first tube end can be operably linked to a proximal end of a first conduit. In another aspect, a second conduit can be disposed on the second end of a tube. In a further aspect, a negative pressure module is disposed on the far end of the second conduit. Whereupon the insertion of a distal first conduit inside an eye, a negative pressure module reduces pressure relative to pressure in an eye thereby creating a vacuum that generates an aspirating action that is transmitted through the tube and via the first conduit into the eye resulting in aspiration of ocular fluid. In an alternative aspect, a tube can be used as a receiving chamber.

In one aspect, a valve can be operably linked to a first or second end of a tube. In another aspect, a valve can regulate the pressure between a low pressure chamber and a tube, a first conduit, a second conduit, or a combination thereof.

In one aspect, an ophthalmic aspirating device further includes a receiving chamber for the ocular fluids. In a preferred aspect, the receiving chamber comprises a material that will reduce the binding of biologics to the receiving chamber. The receiving chamber may further contain one or more stabilizers of biologics. In a preferred aspect, the receiving chamber is constructed in a manner to allow it to be readily transferred to a laboratory for analysis of its contents. For example, the receiving chamber can be constructed to allow its contents to be seen. In another aspect, the receiving chamber is configured to allow an operator to determine the volume of the contents of the receiving chamber. In one aspect, the biologics is one or more biomarkers.

An ophthalmic aspirating device can further comprise a housing. In another aspect, the ophthalmic aspirating device comprises a housing; a tube operatively associated with the housing, the tube having first and second ends; a conduit having first and second ends, wherein the first conduit end comprises an aperture, and where the second conduit end is operatively associated with the first tube end; and a negative pressure module.

In some aspects, a negative pressure module comprises a reservoir operably associated with the housing, where the reservoir comprises at least one chemical to protect polypeptide integrity. In some aspects, the reservoir is comprised by an attachable/detachable receptacle. A negative pressure module can be or can comprise a reservoir or a receptacle. The negative pressure module can be configured for at least one of attachment and detachment from a housing.

Chemicals that can be included in a reservoir, a negative pressure module, housing, or tube include, e.g., protease inhibitors; phosphatase inhibitors; various preservatives, etc. Specific examples include, serine protease inhibitors, cysteine protease inhibitors, aspartic protease inhibitors, and metalloprotease inhibitors. Examples of these include, AEBSF, aprotinin, E-64, EDTA, leupeptin, bestatin, 0-phenanthroline, cathepsin, etc.

According to one aspect of the present invention, a ophthalmic aspirator device includes a tube with two conduits, one connected to each end of the tube. At both ends of the tube, a conduit can be of a very small size, small enough to aspirate very small amounts of liquid, such as, on the order of 20 to 100 microliters. At one end of the tube, a first conduit can be inserted into an eye of a patient. At the other end of the tube, a second conduit can be inserted into a negative pressure module, and a valve can control the communication between the negative pressure module and the tube.

In another preferred aspect, an outer conduit is as described above and an inner tubing has a smaller diameter and is recessed behind the opening of the outer conduit. In one aspect, materials, such as drugs or gas, can be injected though the inner recessed tubing. In a preferred aspect, the conduit is a dual bore needle.

A drug that may be injected through the inner tubing can be bevacizumab (Avastin, Genentech, South San Francisco, Calif.), triamcinolone (Kenalog, Bristol-Meyer Squibb), dexamethasone (American Reagent, Inc. Shirley, N.Y.), ranubizumab, or pegaptanib. In a preferred aspect, less than 500 microliters is injected, preferably less than 200, 100, or 50 microliters is injected.

According to another aspect of the present invention, a syringe can be provided that includes a needle, cannula or other conduit of a very small size at least one end, for example that would allow the aspiration of about 20 to 200 microliters of liquid, of 50 to 100 microliters, and a plunger is provided at the end of the syringe opposite the needle, cannula or other conduit. The plunger, when pulled away from the needle, cannula or other conduit creates a negative pressure module inside the syringe that aspirates fluid in the vicinity of the needle. In one aspect, the syringe includes a biasing device connected to the plunger, and an actuator controls the operation of the biasing device. Accordingly, once the actuator is engaged by a user (e.g., a medical practitioner), the biasing device is activated and urges the plunger of the syringe in a direction away from the eye of the patient so that an aspiration force is created. In operation, the actuator can be arranged so that, once urged, the biasing device pulls the plunger of the syringe away from the eye at a rate sufficient to create a negative pressure module within the syringe that allows the fast extraction of liquid from the eye, without extracting any of the gel present in the vitreous fluid from the eye. As a result, only liquid from the eye is aspirated into the syringe, not any of the biological gel.

According to various exemplary aspects, the needle, cannula or other conduit, or the syringe tube can have markings indicating the amount of fluid being aspirated into the syringe tube, and the user can disengage the actuator once the needle, cannula or other conduit, or the syringe tube indicates that a desired amount of fluid has been aspirated. Once the actuator is disengaged, the biasing device is prevented from further aspirating fluid. Needles and other conduits can have apertures at their ends. One or more of these ends can be sharp to allow puncture of the eye and/or a container such a as a negative pressure module. A secondary housing can be employed to hold at least one of the conduit and the negative pressure module.

Before, after, or concurrent to the removal of fluid from the eye, one or more therapeutic agents can be administered to the eye. The therapeutic agent can be administered using the same conduit used for fluid removal. The therapeutic agent can be housed with the ophthalmic aspiration device. In some aspects the therapeutic agent is housed within a chamber operatively associated with at least one end of the ophthalmic aspiration device. The therapeutic agent can be housed in a container separate from the ophthalmic aspiration device. A chamber or container housing the therapeutic agent can comprise one or more segments. The segments can be separated by one or more septa. A septum can allow passage, e.g., by puncture, by a conduit. Segments can house same or different therapeutic agents. In some aspects, puncturing of one or more septum allows mixing of ingredients for the therapeutic agent. A secondary housing employed to hold at least one of the conduit and the negative pressure module can in addition or in the alternative be used with the therapeutic agent container. In some aspects, the negative pressure module and therapeutic agent container are used in succession, in either order, by removing one and then inserting the other. In some aspects, the therapeutic agent container is an ampoule. The therapeutic agent can be flexible such that when squeezed, the therapeutic agent is released.

Advantages of this invention include that a very small amount of fluid is extracted from the eye of the patient and that the user can use one hand to extract the fluid, thus making extraction easier and more efficient. Accordingly, such fluid extractions can be performed multiple times on a patient without significant adverse effects, such as once every other week, once a month, bimonthly, or on an as needed basis, and can be performed as an outpatient procedure in a doctor's office.

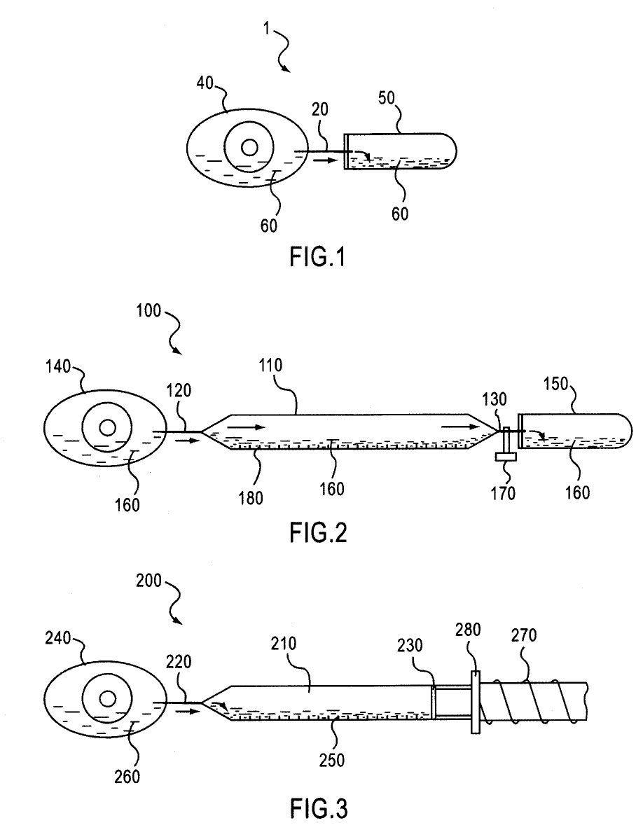

FIG. 1 illustrates an ophthalmic aspiration device 1, according to one exemplary aspect of the invention. In FIG. 1, a conduit 20, such as a needle or cannula, has first and second ends with apertures at the respective ends. The conduit can be operatively associated with at least one of an eye 40 and a negative pressure module 50. Such operative association allow for transfer, e.g., aspiration, of fluid 60 (e.g., vitreous fluid) from the eye 40 to the negative pressure module 50.

An ophthalmic aspiration device can be operated in a manner similar to that used for drawing blood from veins. That is a conduit can have two sharp ends, one for entering the eye and the opposite end for puncturing a stopper in a negative pressure module. By applying pressure to a negative pressure module, the stopper presses against the end of the needle opposite the end in the eye, thereby puncturing the stopper and causing a negative pressure in the module to withdraw vitreous fluid from the eye into the module. As in certain other aspects, a negative pressure module can contain preservatives such as a protease inhibitor. Once the fluid has been removed from the eye, the conduit, optionally with a tube or tubing, can be removed from the eye. Alternatively, the negative pressure module can be removed with conduit in place and a non-vacuum container can replace the negative pressure module on a conduit or on a tube or tubing. A non-vacuum container can house a gas or fluid, for example a drug, to be released into the eye. A non-vacuum container can be made out of a soft material that can be squeezed to push the drug through the conduit into the eye. Once a drug is released, the one or more conduits, or tubing, and module can be removed from the eye. In some aspects, a Vacutainer® tube or system is used.

FIG. 2 illustrates an ophthalmic aspiration device 100, according to one exemplary aspect of the invention. In FIG. 2, a tube 110 is provided with two needles, cannula or other conduits 120 and 130, are at each end of the tube 110. A first needle, cannula or other conduit 120 is connected to the tube 110 at one end and can be inserted inside the eye 140 of a patient via the end opposite the connection to the tube. A second needle, cannula or other conduit 130, located at an end opposite to the first needle, cannula or other conduit 120, communicates with a negative pressure module 150. In operation, when the needle, cannula or other conduit 120 is inserted into the eye 140, and the needle, cannula or other conduit 130 is inserted into the low pressure chamber 150, the reduced pressure in the negative pressure module 150 generates an aspirating action that is transmitted through the tube 150 and via the needle, cannula or other conduit 120 to the interior of the eye 140 or other location to be aspirated. As a result, fluid 160 (e.g., vitreous fluid) present inside the eye 140 is aspirated through the needle 120 and into the tube 110. Alternatively, the fluid 160 can also be aspirated directly into the negative pressure module 150. A certain amount of fluid 160 can be aspirated from the eye 140 of a patient, for example, and into either the tube 110, or into both the tube 110 and the negative pressure module 150. In some aspects, a Vacutainer® tube or system is used.

As an alternative option, a valve 170 can be provided for controlling communication between the negative pressure module 150 and the needle, cannula or other conduit 130, so that once the needle, cannula or other conduit 120 is inserted into the eye 140 of a patient, for example, no aspiration occurs until the valve 170 is opened. Once the valve 170 is opened, the negative pressure module 150 communicates with the tube 110, and via the tube 110 with the needle, cannula or other conduit 120 and the interior of the eye 140. Furthermore, indicators 180 can be present on the side of the tube 110 to allow measurement of the volume of fluid aspirated into the tube 110. The valve 170 can be closed by a user once the amount of fluid 160 aspirated reaches a desired volume. Thus, a better control of the volume of fluid 160 that is aspirated can be achieved.

According to an exemplary aspect of this invention, the tube 110 and the needles, cannulae or other conduits 120 and 130 can be reusable together or separately. Once the user has extracted fluid 160 from a patient's eye, and once the fluid 160 has been transferred to a storage area or has been analyzed, the tube and needle, cannula or other conduit can be cleaned, sterilized and reused with another patient. For the tube 110 and the needles, cannulae or other conduits 120 and 130 to be reusable, additional components, such as a hub and a ferrule, can be used with the needles, cannulae or other conduits 120 and 130. Accordingly, when the user activates the valve 170 to discontinue the aspiration of fluid 160, the valve 170 can incur a small delay, during which an additional volume of fluid 160 can be aspirated beyond the desired amount. For example, the additional volume of fluid 160 can correspond to the volume of fluid present inside the hub and the ferrule of the needles, cannulae or other conduits 120 and 130. Any losses that would have been due to the existence of the hub and the ferrule on the syringe can be compensated, and a precise amount of fluid 160 can be extracted from the inside of the eye 140 of the patient, into the tube 110.

One aspect of various aspects of this invention comprises the aspiration of fluid 160 from the eye 140 very quickly as the aspirating action is caused by communication with the reduced pressure inside the negative pressure module 150. As the vitreous fluid inside the eye 140 can contain both fluid and gel, and because of the quick aspiration action created by communication with the negative pressure module 150, only the fluid portion 160 of the vitreous fluid inside the eye 140 can be aspirated into the tube 110, into the negative pressure module 150, or both.

The amount of fluid aspirated from the inside of the eye 140 can be minimal if the negative pressure module used is relatively small. For example, volumes of about 20 to 100 microliters can be aspirated, or volumes of about 50 to 100 microliters. Also, because very small amounts of fluids can be aspirated from the inside of the eye 140, the procedure can be performed without irrigating the eye or replenishing the aspirated fluid with antibiotics and/or other fluids, as is generally the case for conventional techniques.

Another advantage of some aspects of the present invention is that a medical practitioner can extract liquid from a patient's eye in a routine outpatient procedure using one hand and in a fairly short amount of time.

FIG. 3 illustrates an ophthalmic aspiration device, according to another exemplary aspect of the present invention. In FIG. 3, a tube 210 is provided with a needle, cannula or other conduit 220 at one end, and a plunger 230 at the other end. The plunger 230 can be activated via a biasing mechanism 270 such as a spring or other energy storing mechanical or electrical device. The biasing mechanism 270 can be controlled by an actuator 280 that urges the biasing mechanism 270. As such, when the actuator 280 is engaged by a user, the biasing mechanism 270 is activated, and the plunger 230 travels away from the needle 220, thus creating an aspirating force. When the engaging mechanism 280 is released, operation of the biasing mechanism 270 is arrested, and the plunger 230 is placed in a stationary state. The tube 210 can include indicators 250 indicating the volume of fluid that has been aspirated into the tube 210.

In operation, the needle, cannula or other conduit 220 can be inserted inside the eye 240 of a patient. If the plunger 230 is in a stationary state, then no aspiration of the fluid 260 from the eye 240 can occur. However, when a user engages the urging mechanism 280 to engage the biasing mechanism 270, the plunger 230 is pulled away at a high rate from the needle 220, and an aspiration force is created. Accordingly, the fluid 260 in the eye 240 of a patient can be aspirated inside the tube 210. Once the aspiration of the fluid 260 inside the tube 210 has started, the user can have several options. As a first option, the user can simply arrest the urging mechanism 280 to deactivate the spring biasing mechanism 270 when the aspirated fluid has filled the tube 210. As a second option, the user can monitor the volume of fluid that is being aspirated from the eye 240 of the patient via the indicators 250. Once the fluid reaches a desired volume, the user can then disengage the urging mechanism 280 to arrest the biasing mechanism 270 and to render the plunger 230 stationary, thus terminating the aspirating action. Accordingly, the user is able to aspirate very small amounts of fluid, for example in the range of 20 to 200 microliters, or in the range of 50 to 100 microliters.

Because the vitreous fluid inside of the eye 240 can contain not only fluid 260, but also gel-like substances, the fluid 260 from the inside of the eye 240 can be aspirated by having a high rate of aspiration from the plunger 230. The biasing mechanism 270 can pull away the plunger 230 at a high rate to allow the aspiration of the fluid 260 in a rapid burst of suction, and to leave any gel-like substance inside the eye 240. Once the plunger 230 is pulled away rapidly, the fluid 260 fills the tube 210 more slowly than the rate at which the plunger 230 is pulled, and the user can monitor the fluid 260 and disengage the urging mechanism 280 to stop the plunger 230 when the fluid 260 reaches a desired volume. Because very small amounts of fluids can be aspirated from the eye 240, irrigating the eye or replenishing the aspirated fluid with antibiotics and other fluids can be avoided. Another advantage of some aspects of the present invention is that a medical practitioner can extract fluid from a patient's eye in a routine outpatient procedure using one hand and in a fairly short amount of time.

According to an exemplary aspect of this invention, the tube 210 and the needle, cannula or other conduit 220 can be reusable together or separately. Thus, once a user has extracted fluid 260 from a patient's eye 240, and once the fluid 260 has been transferred to a storage area or has been analyzed, the tube and needle, cannula or other conduit can be cleaned, sterilized and reused with another patient. For the tube 210 and the needle, cannula or other conduit 220 to be reusable, additional pieces such as a hub and a ferrule can be added between the needle, cannula or other conduit 220 and the tube 210. Accordingly, when the user engages the biasing mechanism 270 via the engaging mechanism 280 to discontinue pulling the plunger 230 away from the needle, and thus to terminate the aspiration of the fluid 260, the urging mechanism 280 can include a small delay during which the biasing mechanism 270 is still pulling the plunger 230 to aspirate an additional volume of fluid 260, wherein the additional volume of fluid 260 corresponds to the volume of fluid 260 that is present inside the hub and the ferrule. Thus, any losses that may be due to fluid 260 remaining inside the hub and the ferrule can be compensated, and precise amounts of fluid 260 can be extracted into the tube 210.

Kits including one or more material of the present invention are provided that can be used to carry out the methods of the invention in whole or part. Kits for analyzing vitreous fluid samples are provided. A vitreous fluid analysis kit can comprise a vitreous fluid receptacle comprising a reservoir, wherein the reservoir comprises at least one chemical to protect polypeptide integrity. In some aspects, the vitreous fluid analysis kit further comprises an ophthalmic aspirating device, comprising a housing to which the receptacle can be operatively attached and detached. The vitreous fluid analysis kit can further comprise at least one vitreous fluid biomarker detector. In some aspects, the detector comprises a primary antibody specific to a biomarker polypeptide or a unique fragment thereof In some aspects, the detector comprises a secondary antibody coupled to a label, such as a radioactive or fluorescent label. A variety of other detectable labels are known to those skilled in the art. A kit can include instructions.

A method of remote vitreous fluid analysis is provided. A method comprises obtaining a vitreous fluid sample from a living subject; storing the vitreous fluid sample in a receptacle comprising a reservoir, where the reservoir comprises at least one chemical to protect polypeptide integrity; and sending the vitreous fluid sample to a laboratory for analysis. Once received, a vitreous fluid sample can be analyzed. In some aspects, a analyzing comprises detecting a level of a biomarker in the vitreous fluid, where the biomarker is associated with at least one of a susceptibility to an ocular condition, presence or absence of an ocular condition, and an efficacy of treatment of an ocular condition; and identifying at least one of the susceptibility to the ocular condition, the presence or absence of the ocular condition, or the efficacy of treatment of an ocular condition based on the level of the biomarker. An analysis report can be returned to the sender or a third party. In some aspects, the method comprises selling and/or purchasing a kit of the present invention. The contractual relationship can require the ophthalmology clinic to buy at least one class of articles from the vitreous fluid analysis organization or a supplier designated by the organization. In some aspects, the class of articles is a vitreous fluid analysis kit comprising a vitreous fluid receptacle comprising a reservoir, wherein the reservoir comprises at least one chemical to protect polypeptide integrity.

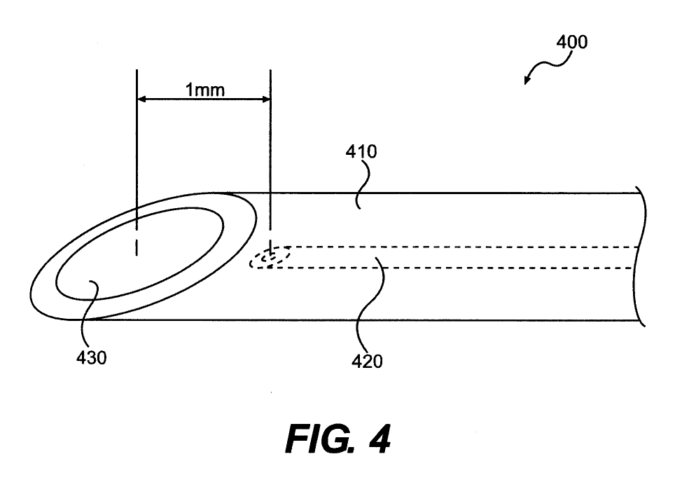

FIG. 4 illustrates a dual bore needle 400 according to an exemplary aspect of the invention. In FIG. 4, an outer needle 410 is provided that has the characteristics of the conduit previously described. In addition, an inner tubing 420 is provided inside of the outer needle 410. The inner tubing 420 has a diameter that is smaller than the diameter of the outer needle 410. Further, the inner tubing 420 is recessed behind the opening 430 of the outer needle. In a preferred embodiment, the inner tubing 420 is recessed one millimeter behind the opening 430 of the outer needle. This allows the injection of materials through the inner recessed tubing 420 into the eye.

An inner tubing can be made of any material. In one aspect, the inner tubing is made of plastic. In another aspect, the inner tubing is made of metal. An inner tubing can be flush with the opening of the needle, but is more preferably behind the opening of the needle. The inner tubing can be 0.5 mm-4 mm behind the opening of the outer needle, preferably 0.5 mm-2 mm, more preferably about 1 mm. An inner tubing can be anywhere within the outer needle. In one aspect, an inner tubing touches the wall of the outer needle. In another aspect, an inner tubing is not touching the wall of the outer needle, preferably in about the center of the outer needle, most preferably in the center of the outer needle.

FIG. 5 illustrates a conduit 520 with a beveled edge 510. In FIG. 5, the angle 530 of the beveled edge is approximately 45 degrees. In a preferred aspect, the angle of the beveled edge is maximized to reduce patient discomfort, to minimize scleral would size, and to allow maximal self sealing of the scleral wound. In a preferred embodiment, the angle of the beveled edge is between 20 and 45 degrees. In a most preferred embodiment, the angle of the beveled edge is 30 degrees. The angle of the beveled edge is measured from a point at the center of the aperture 560 to the bottom of the conduit.

In a further embodiment, the outer edge of the bevel 540 may be sharpened to minimize patient discomfort and maximize ease of passage through the sclera. In still a further embodiment, the inner edge of the bevel 550 may be sharpened to cut vitreous when aspiration is performed under a vacuum, thereby facilitating removal of vitreous and other ocular fluids.

The present application further includes a method for aspirating vitreous fluid from an eye comprising inserting a distal end of a conduit into the eye, where the distal conduit end comprises a first aperture with a beveled edge of between 25 and 40 degrees, and the conduit has an outside diameter between 23 and 27 gauge, an inside diameter between 0.0120 inch and 0.0140 inch, and a wall thickness less than or equal to 0.004 inches; applying pressure to a negative pressure module containing a stopper, where the negative pressure module is operably associated with a proximal end of the conduit containing an aperture, causing the stopper to press against the proximal end of the conduit, thereby puncturing the stopper; creating a vacuum in the negative pressure module between 500 mmHg and 1500 mmHg; and withdrawing between 10-200 microliters of vitreous fluid from the eye into the negative pressure module.

The present application also includes a method for aspirating liquid from an eye comprising: inserting a first conduit into an eye, where the first conduit has an outside diameter between 23 and 27 gauge, an inside diameter between 0.0120 inch and 0.0140 inch, a wall thickness less than or equal to 0.004 inches, an aperture, a beveled edge of between 25 degrees and 40 degrees, and where the first conduit is connected to a first end of a tube; opening a valve, where the valve is located on a second end of the tube, which is connected to a negative pressure module via a second conduit, and where the opening allows the tube to communicate, via the second conduit, with the negative pressure module, resulting in a vacuum of between 500 mmHg and 1500 mmHg inside the negative pressure module; creating an aspiration force inside the tube; and aspirating between 10-200 microliters of vitreous fluid from the eye into the tube.

The present invention further includes a method for aspirating liquid from an eye comprising: inserting a conduit into an eye, where the conduit has an outside diameter between 23 and 27 gauge, an inside diameter between 0.0120 inch and 0.0140 inch, a wall thickness less than or equal to 0.004 inches, an aperture with a beveled edge of between 25 degrees and 40 degrees and the conduit is connected to a first end of a tube; and activating a biasing mechanism, where the biasing mechanism is operably linked to a plunger that is operably linked to a second end of the tube, and the activating causes a pulling of the plunger away from the conduit that creates a vacuum of between 500 mmHg and 1500 mmHg inside the tube; and aspirating between 10-200 microliters of liquid through the conduit into the tube.

The present invention also includes a method for aspirating ocular fluids and for injecting materials into an eye comprising: inserting an outer conduit into an eye, where the outer conduit comprises an aperture, a beveled edge of between 25 and 40 degrees, the outer conduit having an outside diameter between 23 and 27 gauge, and a wall thickness less than or equal to 0.004 inches; applying a vacuum of between 500 mmHg and 1500 mmHg; aspirating between 10-200 microliters of vitreous fluid from the eye; injecting materials into the eye by releasing the materials through an inner tube, the inner tube having an outside diameter between 30-40 gauge, and a wall thickness less than or equal to 0.004 inches, the inner tube disposed in the center of the outer conduit beginning 1 mm or more behind the aperture of the outer conduit.

In accordance with one aspect of the invention, an ophthalmic aspirating device is provided. In one aspect, the ophthalmic aspirating device has negative pressure module and a conduit having a first conduit end and a second conduit end. The first conduit end includes a first aperture and the second conduit end comprises a second aperture the second conduit end is operatively associated with the negative pressure module.

In accordance with yet another aspect of the invention, an apparatus for aspirating ocular fluids in provided. In one aspect, the apparatus for aspirating ocular fluids includes a conduit having a first end with a first aperture for insertion into an eye and a second end with a second aperture. The apparatus also includes a negative pressure module with a stopper linked to the second end. Upon application of pressure to the negative pressure module, the stopper is pressed against the second end causing the second end to puncture the stopper and cause negative pressure in the negative pressure module to aspirate vitreous fluid from the eye into said negative pressure module.

Also included in the present invention are methods for aspirating liquid from an eye. In one aspect, the method for aspirating liquid from an eye including inserting a first end of a conduit into an eye, where the conduit is connected to a second end of a tube. The method also includes opening a valve, where the valve is located on a first end of the tube, which is connected to a negative pressure module via a first conduit, and where the opening allows the tube to communicate, via the first conduit, with a vacuum inside the negative pressure module. The method also includes creating an aspiration force inside said tube.

In one aspect, an ophthalmic aspirating device, comprises: a negative pressure module; a conduit having first and second ends, wherein the respective ends each comprises an aperture, and wherein the second conduit end is operatively associated with the negative pressure module. In another aspect, the ophthalmic aspirating device comprises a housing; a tube operatively associated with the housing, the tube having first and second ends; a conduit having first and second ends, wherein the first conduit end comprises an aperture, and wherein the second conduit end is operatively associated with the first tube end; and a negative pressure module.

Kits for analyzing vitreous fluid samples are provided. A vitreous fluid analysis kit can comprise a vitreous fluid receptacle comprising at least one reservoir, wherein the reservoir comprises at least one chemical to protect polypeptide integrity.

A method of remote vitreous fluid analysis is provided. The method comprises obtaining a vitreous fluid sample from a living subject; storing the vitreous fluid sample in a receptacle comprising a reservoir, wherein the reservoir comprises at least one chemical to protect polypeptide integrity; and sending the vitreous fluid sample and/or data collected therefrom to a laboratory or equivalent facility for analysis. Once received, the vitreous fluid sample can be analyzed. In addition or in the alternative, data collected from the vitreous fluid sample can be sent for analysis. Aspiration can be carried out by any method including without limitation a method for injecting gas that is changed to remove vitreous fluid rather than inject gas, such as the methods set forth in Ryan, S. J., Hinton, D. R., Schachat, A. P., Wilkinson, C. P. (2006). Retina edition, (4th Edition), Mosby, page 2076, herein incorporated by reference.

### EXAMPLE 1

A prospective, consecutive case series is carried out and it included all patients who undergo in-office diagnostic vitreous sampling. All injections and samples are preceded by a standard dilated vitreo-retinal examination which includes a detailed medical history, best-corrected visual acuity, intra-ocular pressure, and detailed dilated retinal exam. Patients with choroidal neovascularization receive intravenous flourescein angiography, indocyane green angiography, and optical coherence tomography. Patients with retinal vascular disease receive intravenous flouresecein angiography and optical coherence tomography (OCT). Inclusion criteria include patients with vitreo-retinal pathology that required intravitreal medication injection (anti-VEGF or corticosteroid) or tap that had greater of thirty days of follow-up. Exclusion criteria are as follows: 1) patients unwilling or unable to consent to study or follow-up; 2) ophthalmic surgery within the last three months; 3) active intraocular inflammation.

The prospective, consecutive case series included diagnostic vitreous sampling. A diagnostic vitreous sample is labeled as positive if greater than 0.05 ml of vitreous fluid is collected. A sample is labeled as “dry” if less than 0.05 mL of vitreous is obtained.

Diagnostic Vitreous Sampling (DVS) is performed before an injection of either bevacizumab 0.05 mL/1.25 mg (Avastin, Genentech, South San Francisco, Calif.), triamcinolone 0.10 mL/4.0 mg (Kenalog, Bristol-Meyer Squibb), or dexamethasone 0.2 mL/800 μg (American Reagent, Inc. Shirley, N.Y.). In one patient (CRAO) a sample is obtained without intravitreal injection.

Vitreous samples are obtained by using a standard technique on all patients as follows: Topical anesthesia is achieved by instillation of topical lidocaine gel 2% (Xylocaine® AstraZeneca LP, Wilmington, Del.) followed by placement of a soaked cotton-tip applicator (lidocaine 4% solution, Roxane Laboratories, Columbus, Ohio) in the inferior temporal quadrant. Using sterile gloves a sterile lid speculum is placed followed by a drop of betadine 5%. Next, a specialized twenty-five gauge needle is placed via pars plana approach into the mid-vitreous cavity and 0.05 to 0.10 mL of vitreous fluid is aspirated into a 1 mL syringe (PrecisionGlide® Becton Dickinson, Franklin Lakes N.J.). Following removal of the needle, a thirty gauge needle (PrecisionGlide® Becton Dickinson, Franklin Lakes N.J.) is introduced into the same relative area and the injection performed. Intraocular pressure is only measured if a vitreous sample is not obtained (“dry sample”). After injection, the patient is discharged with post-injection warnings and instructions to use a fourth generation flouroquinolone antibiotic drop (moxifloxacin or gatifloxicin) four times a day for five days. Patients are educated regarding the warning signs that would require immediate evaluation. Patients are followed at intervals of one month. Safety and adverse events are documented at all follow-up visits using a standardized history and examination questionnaire completed by the examining physician. Common injection related findings such as subconjunctival hemorrhage and localized injection discomfort are not recorded. All other intraocular changes are noted on safety and adverse event forms.

A total of 550 positive diagnostic vitreous samples are collected from 157 eyes of 144 patients. There are 49 males and 95 female participants. The average age of the subjects is 77.3 (range 33-98). There are 86 phakic eyes and 71 pseudophakic eyes. The minimum number of sample/injections is 1 while the maximum is 12. The average number of samples per eye is 3.5. The average follow-up time is 205 days (range 30-613 days). This data is presented in Table 1.

TABLE 1
Subject Demographics

| | |
|---|---|
| Number of Subjects | 144 |
| Number of Eyes | 157 |
| Total Number of Samples | 550 |
| Male | 49 |
| Female | 95 |
| Average Age | 77.3 |
| Age Range | 33-98 |
| Average number of samples | 3.5 |
| Average follow-up time (days) | 205 |
| Follow-Up Period Range (in | 30-613 |
| days) | |

The vitreo-retinal diagnoses includes six categories as follows: exudative age-related macular degeneration (n=134), proliferative diabetic retinopathy/vitreous hemorrhage (n=13), venous occlusive disease (CRVO/BRVO) (n=8), diabetic macular edema (n=1 ), central retinal artery occlusion (n=1), presumed ocular histoplasmosis (n=1). The subject with central retinal artery occlusion is the only patient who undergoes diagnostic vitreous sampling without intravitreal injections (Table 2). None of the patients have history of vitrectomy.

TABLE 2
Vitreo-retinal diagnoses included

| | # of |
|---|---|
| Diagnoses | Eyes |
| Exudative Age Related Macular Degeneration | 134 |
| Proliferative Diabetic Retinopathy/Vitreous | 13 |
| Hemorrhage | |
| Venous Occlusion Disease (CRVO/BRVO) | 8 |
| Central Retinal Artery Occlusion | 1 |
| Presumed Ocular Histoplasmosis Syndrome | 1 |

There are a total of 28 “dry” samples in 19 subjects (13 female, 6 male). The rate of dry sample is 4.8% (28/578). Ten patients were pseudophakic (1 subject with anterior chamber intra-ocular lens, 9 subjects with posterior chamber intraocular lens) while 9 are phakic. All patients are diagnosed with age-related macular degeneration. Five subjects account for 54% of all dry samples (15/28). Eighteen of the nineteen subjects who had experienced a “dry” sample have at least one positive sample in during the series. The average number of previous intra-vitreal injections prior to the dry sample is 3.7 (range 0-10) (Table 3).

TABLE 3
Analysis of Dry Samples

| Dry Samples | |
|---|---|
| Total Number Successful Samples | 550 |
| Total Number of Dry Samples | 28 |
| Percentage of Total | 4.8% |
| | (28/578) |
| Number of patients | 19 |
| Lens Status | 1 |
| ACIOL | |
| PCIOL | 9 |
| Phakic | 9 |
| Average number of samples prior to dry | 3.7 |
| sample | |
| Number of patients with multiple dry samples | 5 (15/28) |

There are a total of 7 complications out of 578 vitreous sampling attempts (1.2%) noted on follow-up examinations. All these subjects have diagnostic vitreous sampling followed by intravitreal bevacizumab (0.05 mL/1.25 mg). There are three types of complications noted: 1) intraocular inflammation, 2) vitreous hemorrhage, 3) chronic, localized retinal detachment (Table 4).

TABLE 4
Types of Complications

| Complications | # of | |
|---|---|---|
| Type | Patients | Sequelae |
| Vitreous hemorrhage | 3 | No |
| Intraocular inflammation | 3 | No |
| Localized Retinal Detachment | 1 | Stable |

Three patients have an intraocular inflammatory response which responds to topical prednisolone acetate 1% drop therapy. All of these cases show resolution of inflammation within a six week period. Of the patients with intraocular inflammation, one has a repeat episode which responds to topical steroid drops. Three patients have mild (1+) vitreous hemorrhage without any retinal breaks or tears noted. All patients have complete resolution of vitreous hemorrhage within eight weeks. One patient is noted on follow-up to have an asymptomatic, localized rhegmatogenous retinal detachment (superior temporal) with a pigmented demarcation line. There is a small retinal hole associated with this retinal detachment. Prophylactic laser indirect ophthalmoscopy is completed and the detachment remains stable on follow-up examination at the two month visit.

In this prospective study, the ability to obtain a safe, positive yield, in-office diagnostic vitreous sample is demonstrated over a large number of subjects. The success rate of a positive yield (95.2%) is accompanied by a low side effect profile of 1.2% (n=7). The main complications are a mild vitreous hemorrhage in 0.005% (n=3) and intraocular inflammation in 0.005% (n=3) both of which resolve within a six week period. Intraocular inflammation is related to the injectable medication (bevacizumab) has been reported in the literature. Despite this, a definitive cause-effect could not be drawn whether the intravitreal injection or the intravitreal sample was responsible for the complication. The mild vitreous hemorrhages in the three subjects could have resulted from either the vitreous sample or injection of medication. It is unknown whether the hemorrhage is sample or injection related. In all three cases the vitreous hemorrhage appears to have no long term sequelae. These side effects are reported in the literature associated with intravitreal injection alone and are comparable to the adverse events reported in the MARINA trial. Furthermore, there have been reports of retinal pigment epithelial rips and endophthalmitis after intravitreal injections—both of which are not seen in this series. In the case of the chronic localized rhegmatogenous retinal detachment, it is also unclear whether a cause and effect relationship exists between the sampling and the detachment. Furthermore, retinal detachment is a known risk with any type of intravitreal injection. There is no clear predictable factor (age, sex, lens status, number of previous injections) for complications.

Over the course of the series, there are 29 dry samples of nineteen patients. Several patients (n=5) have multiple dry taps over the course of the study. This may indicate that a subset of patients may have a vitreous that is more “resistant” to sampling. In this series, detailed vitreous anatomy is not studied with B-scan imaging techniques. A follow-up period of 30-610 days follows.

An added benefit of diagnostic vitreous sampling is the lack of post-injection intraocular pressure spike. Reports in the literature have studied the post-injection pressure spikes associated with bevacizumab, ranubizumab, pegaptanib, and triamcinolone. Although the long term effect of these transient intraocular pressure spikes have not been studied to our knowledge, certain patients may be sensitive to short term pressure spikes. With diagnostic vitreous sampling, there should not be an IOP spike because the injection volume is smaller than the sample volume.

All references, including publications, patent applications, and patents, cited herein are hereby incorporated by reference to the same extent as if each reference were individually and specifically indicated to be incorporated by reference and were set forth in its entirety herein.

What is claimed is:

1. An ophthalmic aspirating device for aspirating an ocular fluid, comprising:
a conduit having an outside diameter between 23 and 27 gauge and a wall thickness less than or equal to 0.004 inches, wherein said conduit has a distal conduit end for insertion into an eye with a first aperture and with a beveled edge of between 25 degrees and 40 degrees, and said distal conduit end being in fluid flow communication with a proximal conduit end with a second aperture; and
a negative pressure module operably linked to said proximal conduit end capable of creating a vacuum of between 500 mmHg and 1500 mmHg for aspiration of between 10-200 microliters of ocular fluid.

2. The ophthalmic aspirating device of claim 1, wherein said wall thickness is 0.004 to 0.002 inches.

3. The ophthalmic aspirating device of claim 1, wherein said ocular fluid is vitreous fluid.

4. The ophthalmic aspirating device of claim 1, further comprising a tube and a second conduit.

5. The ophthalmic aspirating device of claim 1, wherein said beveled edge is about 30 degrees.

6. The ophthalmic aspirating device of claim 1, wherein said conduit has an inside diameter between 0.0120 inch and 0.0140 inch.

7. The ophthalmic aspirating device of claim 1, wherein said conduit has an outside diameter of 25 gauge.

8. The ophthalmic aspirating device of claim 1, wherein said vacuum is 1000 mmHg.

9. The ophthalmic aspirating device of claim 1, wherein said aspirated ocular fluid is 50 microliters.

10. The ophthalmic aspirating device of claim 1, wherein said outside diameter is 0.200 inches.

11. A dual bore conduit for aspirating ocular fluid and for injecting a different fluid into an eye, comprising:
an outer conduit for insertion into an eye, said outer conduit having an outside diameter between 23 and 27 gauge, and a wall thickness 0.002 to 0.004 inches, an aperture having a beveled edge of between 25 degrees and 40 degrees; and
an inner tubing for injecting a different fluid into said eye, said inner tube having an outside diameter between 30 gauge and 40 gauge, said inner tubing disposed in about the center of said outer conduit beginning 1 mm or more behind said aperture of said outer conduit.

12. The ophthalmic aspirating device of claim 11, wherein said outer conduit outside diameter 25 guage.

13. The ophthalmic aspirating device of claim 11, wherein said ocular fluid is vitreous fluid.

14. The ophthalmic aspirating device of claim 11, wherein said beveled edge is about 30 degrees.

15. The ophthalmic aspirating device of claim 11, further comprising a negative pressure module operably linked to said dual bore conduit capable of creating a vacuum of between 500 mmHg and 1500 mmHg.

16. The ophthalmic aspirating device of claim 11, wherein said outer conduit has an outside diameter of 25 gauge.

17. The ophthalmic aspirating device of claim 11, wherein said outer conduit has in inside diameter of about 0.0130 inch.

18. A conduit for aspirating ocular fluid, comprising:
a conduit having an outside diameter between 23 and 27 gauge and an aperture with an beveled edge between 25 and 40 degrees.

19. The conduit for aspirating ocular fluid of claim 18, wherein said conduit has a wall thickness of 0.004-0.002 inches.

20. The conduit for aspirating ocular fluid of claim 18, wherein said conduit has in inside diameter of about 0.0130 inch.
